SEQUENCE LISTING (6 sequences)

SEQ ID NO: 1 - protein, 98 aa, Homo sapiens
  misc_feature (1)..(98): BINDING required for specific binding to recombinant human CD152; SIMILAR human VH3 germline genes.
QVNLRESGGGVVQPGRSLRLSCAASGFTFSSYGMHWVRQAPGKGLEWVAIIYYDGRKIYYADSVKGRFTISRDNSENTVSLQMNSLRAEDTAVYYCAR

SEQ ID NO: 2 - protein, 89 aa, Homo sapiens
  misc_feature (1)..(89): BINDING required for specific binding to recombinant human CD152; SIMILAR human Vof germline genes.
SYVLTQPPSASGTPGQRVTISCSGSSSNIGNNYVYWYQHFPGTAPKLLIYRSKQRPSGVPDRFSGSKSGTSASLAISGLRSEDEADYYC

SEQ ID NO: 3 - DNA, 23 bp, Homo sapiens
  misc_feature (1)..(23): Designed for a primer used in RT-PCR according to length, sequence homology and melting point.
caggtcaacttaagggagtctgg

SEQ ID NO: 4 - DNA, 23 bp, Homo sapiens
  misc_feature (1)..(23): Designed for a primer used in RT-PCR according to length, sequence homology and melting point.
tgagagacggtgaccgtggtccc

SEQ ID NO: 5 - DNA, 23 bp, Homo sapiens
  misc_feature (1)..(23): Designed for a primer used in RT-PCR according to length, sequence homology and melting point.
tcctatgtgctgactcagccacc

SEQ ID NO: 6 - DNA, 24 bp, Homo sapiens
  misc_feature (1)..(24): Designed for a primer used in RT-PCR according to length, sequence homology and melting point.
acctaggacggtgaccttggtccc

(19) United States
(12) Patent Application Publication
(10) Pub. No.: US 2006/0240006 A1
(43) Pub. Date: Oct. 26, 2006

(54) Novel antibody structures derived from human germline sequences

(76) Inventors: Chishih Chu, Taichung (TW); Li-Te Chin, Hsin-Chu (TW); Shu-Ching Hsu, Kaohsiung (TW)

Correspondence Address: PRO-TECTOR INTERNATIONAL SERVICES, 20775 NORADA CT., SARATOGA, CA, 95070, US

(21) Appl. No.: 11/111,098

(22) Filed: Apr. 20, 2005

(51) Int. Cl. G01N 33/567; A61K 39/395; C07K 16/28

(52) U.S. Cl. 424/144.1; 435/7.2; 530/388.22

(57) ABSTRACT

In order to provide necessary information for the production of complete human monoclonal antibodies capable of human CD152 (CTLA-4) binding, the primary structures of heavy and light chains have been elucidated. The novel amino acid sequence of identified heavy and light chains are derived from VH3 and Vλ germline genes, respectively. Antibodies comprising such novel structures cause specific binding to soluble recombinant human CD152 as well as to activated human peripheral T cells, where the expression of CD152 has been elevated.

### BACKGROUND OF THE INVENTION

1. Field of the Invention

The present invention relates to novel primary structures of complete human antibodies and, more particularly, to structures most probably derived from human germline genes and the capability of such structures to specifically bind to human CD152 (CTLA-4) both in solution and on cell surface. Being inclusively originated from human, these structures might ameliorate or even eliminate host response to administrating antibodies commonly found in antibody therapy.

2. Description of Related Art

Immunoglobulins (Igs, antibodies) have been described as Y-shaped proteins on the surface of B cells that are secreted into the blood, lymph and body fluid in response to an antigenic stimulus, such as a bacterium, virus, parasite, or transplanted organ, and they neutralize the corresponding antigen by binding specifically to it. As shown in FIG. 1, it is generally recognized that an antibody structure consists of variable (1a) and constant (1b) regions. There are three hypervariable domains (1f) within each variable region. Amino acids contributed to antigen binding are situated in the hypervariable domain and thus also termed as complementarity determining region (CDR).

Usually, to produce sufficient amount of antibody, the mice are injected or immunized with desired antigen to obtain specific B cells. B cells from euthanized mice are then fused with myeloma to generate hybridoma cell line capable secreting mononoclonal antibodies for an indefinite period. However, the resulting antibodies have murine sequences which, when administered to a human patient, elicit detrimental human anti-mouse immunological responses in the patient thus limit the utility of mouse monoclonal antibodies for therapy. To overcome this problem, humanized antibodies are typically prepared by replacing regions of mouse antibodies that are unimportant for antigen specificity with a human counterpart. To accomplish this particular goal, humanized protocols have been revealed lately. For example, U.S. Pat. No. 5,585,089 discloses how to transfer the binding site (CDRs) of a mouse antibody onto a human one, as well as to introduce amino acid substitutions from the mouse antibody into the framework region of the humanized antibody. In clinical settings, these humanized antibodies have consistently shown minimal human anti-mouse antibody response and have been successfully used for therapeutic drugs against various diseases. These diseases are traditionally infectious diseases, such as infections by respiratory syncytial virus (RSV). Recently, antibodies are increasingly used in the therapy of many other disorders, including autoimmune disorders and malignancies like metastastic breast cancer, non-Hodgkin's lymphoma, chronic lymphocytic leukemia and acute myeloid leukemia. Prophylactic use against organ rejection or blood clotting during angioplasty has also been achieved. However, despite the wealth of successful data accumulating on humanized antibodies, residual murine sequences and adverse effects still exist. Therefore, it is desirable to prepare fully human antibodies that are void of non-human sequences.

By immunizing engineered transgenic mice harboring human immunoglobulin genes, fully human antibodies have indeed been reported. Regretfully, the relatively limited genetic space inherent in an experimental mouse presents significant obstacles to encompass all human immunoglobulin germline genes. As has been discussed by Jakobovits (Curr Opin Biotechnol. 6:561, 1995), the light chain replacement has been restricted to human K germline genes and an entire human repertoire is more difficult to achieve. Although limitation exists, this particular constraint tool still provides a very appealing solution for the production of complete human monoclonal antibodies, as WO 01/14424 documents a CD152-specific antibody derived from a human K germline gene.

The present invention represents a substantiated example and a continuation of U.S. patent application Ser. No.10/866,120 filed on Jun. 22, 2004, which is a continuation of improvement from “site-directed in vitro immunization” technology first conceived and formulated by the inventor (Chin et al. Immunol. 81:428, 1994; Eur. J. Immunol. 25:657, 1995). Techniques of site-directed in vitro immunization are in vitro human lymphocyte stimulation processes to achieve antibody response to a protein antigen by using a fraction of the protein of interest and are known in the art. For example, Zafiropoulos et aL (J Immunol Methods. 200:181, 1997) successfully repeated the preparation, characterization and use of the technology described by the inventor. By using a rather infinite genetic combination and thus, diversity, inherent in human lymphocytes from different individuals, novel structures could be identified. The novelty is at least exemplified by the fact that a distinguished λ germline gene was identified, which is an extremely difficult if not a fundamentally impossible task by using a transgenic animal described above.

### SUMMARY OF THE INVENTION

The object of the present invention is to provide effective, human-originated structural information for producing human antibodies to CD152 without unwanted responses, such as human anti-mouse response or allergic responses.

To achieve the object, the method of the present invention for producing human antibodies comprising following steps: (a) stimulating human lymphocytes with the CD152 immunogens in vitro; (b) identifying and optionally screening the human lymphocytes that produce antibodies able to recognize CD152; and (c) obtaining sequence data from cloned lymphocytes.

The diagram on FIG. 1 shows the primary structure of an IgG antibody, wherein it consists of two heavy and two light polypeptide chains. Unusual properties of diversity cause partially by the presence of variable and constant regions on the same individual polypeptide chain. Additionally, the antigen-binding site, which binds to an epitope and characterized of an antibody, is a cleft formed by folded variable regions of the heavy (VH) and light chains (VL). Sequence analysis of constant regions revealed that all antibodies have one of two kinds of L chain, κ or λ; each antibody has two identical κ chains or two identical λ chains. Similarly, five different H chains have been found: μ, δ, γ, β, and ε.

On the other hand, Ig genes are segmented and can be randomly spliced together. Taking human Igs for example, gene segments encoding Ig H, κ, and λ chains are found on chromosome 14, 2 and 22, respectively. However, Ig gene segments in mammals are not scattered but arranged in groups of variable (V), diversity (D), joining (J), and constant (C) exons (FIG. 2). The variable regions of an antibody protein, which contribute to antigen binding, are encoded by the spliced products of V, (D) and J germline gene segments with V plays the most important role. It is widely accepted that the germline genes of heavy chain can be classified as VH1 to VH7 while the germline genes of light chain can be classified as κ or λ.

In physiological conditions, mutations occur preferentially in the so-called hypervariable CDR regions encoded mainly by the V germline segment. Mutations also occur in the framework regions (FRs) surrounding individual CDR, although less frequent. As the immune response progresses, this “somatic hypermutation” process ensures the average affinity of the antibody produced increases (affinity maturation). The idea of the present invention is thus to exploit the nature of human Ig germline structures for anti-CD152 by using the site-directed in vitro immunization techniques.

Having sequenced VHnovel and VLnovel, a homology search was performed to compare VHnovel and VLnovel to all of the different mammal V genes in a large GenBank database (National Center for Biotechnology Information; NCBI, Washington, D.C.) and to find other homologous proteins by which those sequences in the database with the closest match, or most homology, are reported. Homology searches were accomplished over the www using the program BLAST (Basic Local Alignment Search Tool) from NCBI.

The resultant novel antibody structures derived from human germline genes are amino acid sequences of VH (VHnovel, SEQ ID NO: 1) and VL (VLnovel, SEQ ID NO: 2). As shown in FIGS. 3 and 4, the VH and VL are most probably derived from and most analogous to VH3 and Vk human germline genes, respectively. By comparing the VHnovel result with the available Ig sequences, we conclude that the VHnovel (SEQ ID NO: 1) may be associated with an allelic form of human VH3 germline segment which with genes of accession number AB019439, VH3-30 and VH3-30 being 89.80% (88/98) identity (FIG. 3). Alignments have also disclosed homology of VLnovel (SEQ ID NO: 2) to existing human Vλ germline genes with a measure of 92.13% similarity to genes of accession number BAC01778, S78058 and CAA38313 (FIG. 4). High similarity to accessible V germline genes of human but not others origin is evidence for complete human antibody.

In addition to the human origin confirmed by the homology algorithm, VHnovel and VHnovel corroborate specific binding to recombinant human CD152 (FIG. 5). Furthermore, antibodies comprising such novel structures cause specific binding to activated human peripheral T cells, where the expression of CD152 has been elevated (FIG. 6).

### BRIEF DESCRIPTION OF THE DRAWINGS

FIG. 1 is a schematic representation of a well-recognized IgG structure.

FIG. 2 is the gene construction profiles of human Ig heavy chains.

FIG. 3 shows alignments of VHnovel (SEQ ID NO: 1) to known human VH germline sequences of the highest homology scoring.

FIG. 3 shows alignments of VHnovel (SEQ ID NO: 1) to known human VH germline sequences of the highest homology scoring.

FIG. 4 shows alignments of VLnovel (SEQ ID NO: 2) to known human VL germline sequences of the highest homology scoring.

FIG. 5 represents ELISA reactivity profiles of a novel structure-containing human antibody. The specimen was ten-fold serially diluted and used to evaluate the performance of specificity.

FIG. 6 illustrates flow cytometry analysis of CD3⁺ T cells expressing CD152.

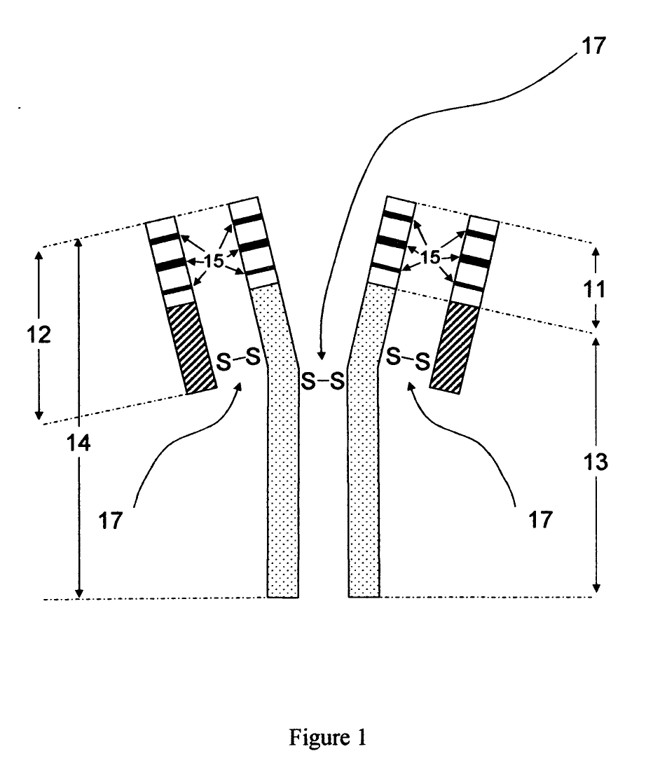

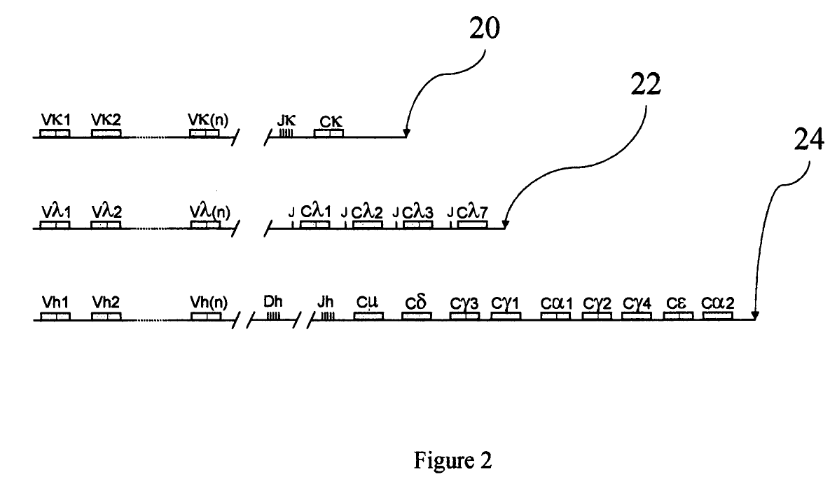

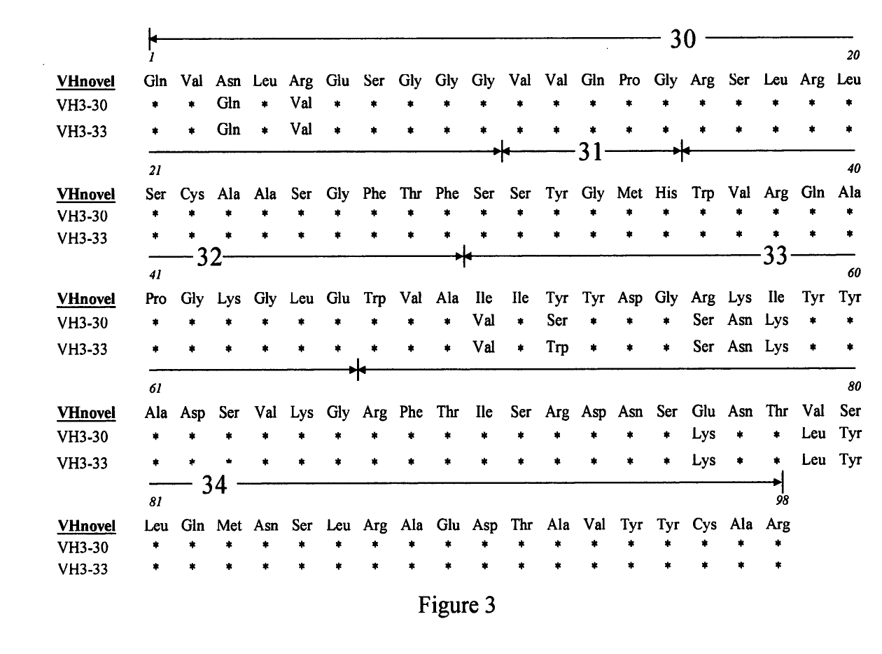

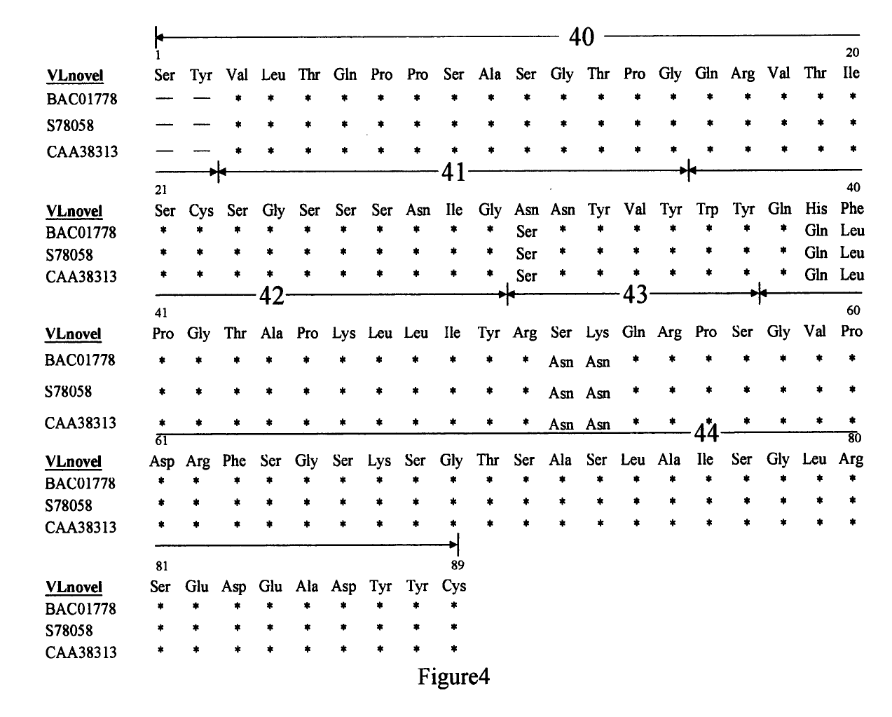

### SYMBOLS USED IN THE DRAWINGS

| | |
|---|---|
| 11: Variable region | 12: Light chain |
| 13: Constant region | 14: Heavy chain |
| 15: Hypervariable region | 17: Disulfide bond |
| 20: Kappa (κ) light chain | 22: Lambda (λ) light chain |
| 24: Heavy chain | 30: FR1 of VH |
| 31: CDR1 of VH | 32: FR2 of VH |
| 33: CDR2 of VH | 34: FR3 of VH |
| 1˜98: Amino acid sequence of VH | |
| *: Amino acid identity to the novel gene | |
| 40: FR1 of VL | |
| 41: CDR1 of VL | 42: FR2 of VL |
| 43: CDR2 of VL | 44: FR3 of VL |
| 1˜89: Amino acid sequence of VL | |
| —: Amino acid deletion to the novel gene | |
| ●: Human CD 152 | □: Monoclonal |
| | murine IgG2a |
| ⋄: Bovine serum albumin | ▾: Tetanus |
| | toxoid |
| 60: Labeling of resting CD3⁺ T | |
| cells using an isotype-matched but | |
| irrelevant IgG plus FITC labeled secondary | |
| antibody. | |
| 62: Labeling of resting CD3⁺ T | |
| cells using an IgG composed of novel | |
| structures plus FITC labeled secondary | |
| antibody. | |
| 64: Labeling of activated CD3⁺ T | |
| cells using an isotype-matched but | |
| irrelevant IgG plus FITC labeled | |
| secondary antibody. | |
| 66: Labeling of activated CD3⁺ T cells | |
| using an IgG composed of novel | |
| structures plus FITC labeled | |
| secondary antibody. | |

### DETAILED DESCRIPTION OF THE PREFERRED EMBODIMENT

The present invention provides information of preparing fully human antibodies that recognize CD152 as the specific antigen. To this end, lymphocytes from naive human donors are immunized in vitro with CD152 immunogens, and cells that produce antibodies against the antigen are identified, selected and sequenced.

This invention also includes pharmaceutical compositions that contain, as the active ingredient, one or more of the antibodies or fragments thereof in combination with a pharmaceutically acceptable carrier or excipients. In preparing the compositions of this invention, the active ingredient/antibody/fragment thereof is usually mixed with an excipient, diluted by an excipient or enclosed within such a carrier, which can be in the form of a capsule, sachet, paper or other container. When the pharmaceutically acceptable excipient serves as a diluent, it can be a solid, semi-solid, or liquid material, which acts as a vehicle, carrier or medium for the active ingredient. Thus, the compositions can be in the form of solutions (particularly sterile injectable solutions), tablets, pills, powders, lozenges, sachets, cachets, elixirs, suspensions, emulsions, syrups, aerosols (as a solid or in a liquid medium), ointments containing, for example, up to 10% by weight of the antibody, soft and hard gelatin capsules, suppositories, and sterile packaged powders.

The following examples are offered to illustrate this invention and are not to be construed in any way as limiting the scope of the present invention.

### EXAMPLE 1

### Generation of Anti-CD152 Human Antibodies

Buffy coats from healthy blood donors, screened negative for HIV-1/2, HTLV-I/II, HCV, HBsAg and containing normal levels of alanine transferase (ALT), were obtained from the Hualien Blood Center, Chinese Blood Services Foundation (Hualien, Taiwan). Peripheral blood mononuclear cells (PBMC) were isolated by density centrifugation (400×g) on Ficoll-Paque (GE Healthcare, Uppsala, Sweden).

The obtained PBMC were first magnetically labeled with CD45RO MACS microbeads (Miltenyi Biotec, Auburn, Calif.) then separated by using a VarioMACS (Miltenyi Biotec) instrument. The eluted CD45RO⁺ cells were recovered by 100×g centrifugation and were used immediately in culture at a density of 2×10⁶ cells/ml in RPMI-1640 (HyQ™; HyClone, Logan, Utah) supplemented with 1× non-essential amino acids (Life Technologies, Grand Island, N.Y.), 10% human serum, 50 μg/ml gentamycin/kanamycin (China Chemical & Pharmaceutical, Taipei, Taiwan), 50 μM 2-mercaptoethanol and 10 μg/ml pokeweed mitogen (PWM; Sigma Chemicals, St. Louis, Mo.). After 24 hr incubation, cells were spun down and removed by 400×g centrifugation. Finally, CD45RO⁺ T cell replacing factor, i.e., culture supernatant, was prepared by harvesting the culture supernatant, filtering with a 0.45 mm filter, and stored frozen at −20° C.

Magnetic cell depletion was performed on PBMC to remove cytotoxic cell populations, which inhibit in vitro immunization. Colloidal super-paramagnetic microbeads conjugated to monoclonal anti-mouse CD8 and anti-CD56 antibodies (Miltenyi Biotech) were used as described above. Cytotoxic cell-depleted PBMC, were immunized in vitro using a two-step immunization protocol. Primary immunization was performed by incubating the cells for 6 days in a medium containing CD152 immunogens and 50 μM 2-mercaptoethanol, 10% heat-inactivated human serum, 0.05 ng/ml rIL2 (Calbiochem, San Diego, Calif.), and 25% (v/v) CD45RO⁺ T cell replacing factor. On day 7, cells from the primary immunization were harvested and spun through 40% Ficoll-Paque. For secondary immunization, 3×10⁷ cells were mixed with CD152 immunogens in a flask that had been immobilized overnight with 5 μg/ml of CD40L (CD154; Vinci-Biochem, Vinci, Italy). The cells were cultured for 3-5 days in a medium supplemented with 5% human serum, 50 μM 2-mercaptoethanol and 10 nM peptide antigen.

The in vitro immunized cells were then infected with EBV Briefly, 10⁷ lymphocytes were incubated for 2 hr at 37° C. with occasional resuspension with 1 ml EBV-containing supernatant derived from the EBV-producing marmoset cell line B95-8 (American Type Culture Collection, ATCC CRL 1612; kindly provided by Dr. L.-F. Shu, Tri-Service General Hospital, Taipei). The infected cells were seeded at 10⁵/well in 96-well plates together with mytomycin (Kyowa Hakko Kogyo, Toyoko, Japan)-treated PBMC as feeder cells (10⁴/well). CD152 reactivity was confirmed by antigen-specific enzyme-linked immunosorbent assays (ELISA).

### EXAMPLE 2

### ELISA Profiling of Anti-CD152 Human Antibodies

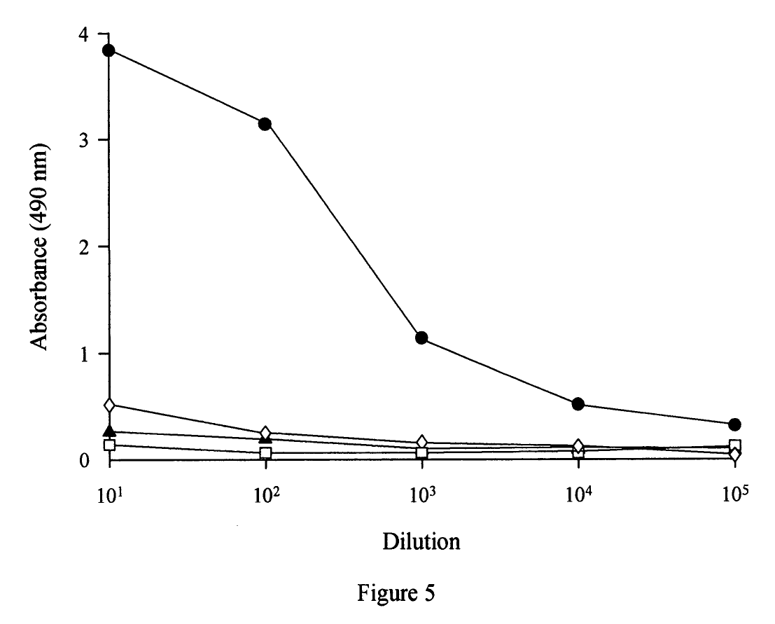

ELISA was performed by first coating 1 μg/ml BHK cell-expressed recombinant human CD152 (CTLA-4)-mulg fusion protein (Ancell Corporation, Bayport, Minn.), 1 μg/ml monoclonal murine IgG2a (Ancell), 10 μg/well of bovine serum albumin (BSA; Sigma) or tetanus toxoid (TT, ADImmune Corporation, Taichung, Taiwan) onto microtitre plates overnight at room temperature. Culture supernatants were diluted to the desired level in 10 mM sodium phosphate buffer, pH 8.0, containing 0 5 M sodium chloride and 0.1% Tween-20. Coated plates were incubated with diluted culture supernatants, washed, incubated with peroxidase-labeled goat antibodies against human IgG (Zymed Laboratories, So. San Francisco, Calif.) and developed (15 min) by addition of 100 μl of the chromogenic substrate o-phenylaenediamine (OPD) (Sigma). The reaction was stopped after 30 min by adding 1 M sulphuric acid, and the absorbances were read at 490 nm. EBV-infected lymphoblastoid cells secreting putative anti-CD152 antibodies were identified and cloned by limiting dilution. As shown in FIG. 5, the identified monoclonal antibody responded specifically to CD152 but unrelated antigens such as murine IgG2a, BSA and TT.

### EXAMPLE 3

### Novel Structures Identification

The novel antibody primary structures were deduced by cDNA sequencing from cloned anti-CD 152-specific cells. Briefly, poly(A)⁺ RNA was isolated from 2×10⁴ cells by using Dynabeads® mRNA DIRECT™ Micro Kit (Dynal Biotech, Oslo, Norway). Purified mRNA was then employed as the reaction template in reverse transcription polymerase chain reactions (RT-PCR). The RT-PCR was carried out with Titan One Tube RT-PCR System (Roche Diagnostics Corporation, Indianapolis, Ind.). PCR primer sets (1 μM) used to amplify human VH and VL were HuVH-JH (SEQ ID NO: 3 and 4) and HuVλ (SEQ ID NO: 5 and 6), respectively. The 37 temperature cycles include: one 2-min denature cycle of 94° C.; 35 cycles of 3-min denaturation at 94° C., 30-sec annealing at 51° C. and 1-min extension at 68° C.; and a final 10-min extension cycle of 68° C. Single banded PCR fragments confirmed by agarose gel electrophoresis were subjected to nucleotide sequencing. Sequences were verified (Molecular Clinical Diagnostic Laboratory, DR. Chip Biotechnology, Inc., Taipei, Taiwan) and converted to amino acids.

### EXAMPLE 4

### Interaction of Novel Structures with Human T Cells

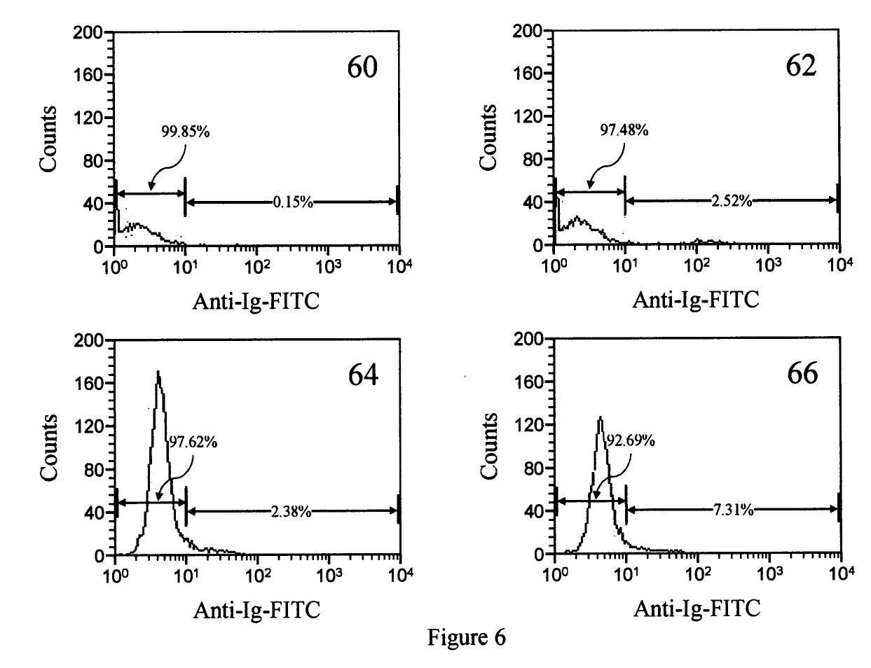

To further investigate the binding specificity of the human anti-CD152 antibody on cellular surface of human peripheral T lymphocytes stimulated in vitro, cultures of PBMC that proven to elevate CD152 surface expression were established. Briefly, 10-ml cultures containing 2×10⁶ cells/ml, 10 μg/ml phytohemagglutinin (PHA; GE Healthcare), 10 ng/ml phorbol 12-myristate 13-acetate (PMA; Sigma), 10% autologous plasma and RPMI-1640 medium were incubated in a humidified atmosphere of 5% CO₂ in air at 37° C. for 72 h. Two-color flow cytometry on the resultant cells to detect surface expression of CD152 was performed using a FACSCalibur flow cytometer (Becton Dickinson Immuno-cytometry Systems, Mountain View, Calif.), interfaced to a Macintosh computer. Data analysis was performed using Cell Quest software (Becton Dickinson). Logarithmically amplified fluorescence data were collected on 10,000 CD3 cells. All flow cytometry staining procedures were performed at 4° C. in flow cytometry buffer (13 PBS, 0.01% NaN₃, 1% BSA; Sigma). For extracellular detection of CD152, activated cells were first surface stained using anti-CD3-PE mabs and the novel human anti-CD152 or isotype control at 4° C. and stained with anti-human IgG-FITC. The results in FIG. 6 indicate that the novel human anti-CD152 stains preferentially to activated CD3⁺ T cells (7.31% vs. 2.38%) where CD152 is expressed at higher levels. This result is representative of four independent experiments.

Although the present invention has been explained in relation to its preferred embodiment, it is to be understood that many other possible modifications and variations can be made without departing from the spirit and scope of the invention as hereinafter claimed.

What is claimed is:

1. Human antibody capable of human CD152 (CTLA-4) binding or an antibody fragment thereof, wherein the heavy and light chain of said antibody is derived from VH3 and Vλ human germline gene, respectively.

2. Use of antibody structures as claimed of claim 1 to diagnose or treat human diseases cause by over- and/or under-expression of CD152.

3. The antibody structures as claimed in claim 1, wherein the amino acid sequence of heavy chain has at least 70% amino acid sequence identity to that of SEQ ID NO. 1.

4. The antibody structures as claimed in claim 1, wherein the amino acid sequence of light chain has at least 70% amino acid sequence identity to that of SEQ ID NO. 2.
